(19) United States
(12) Patent Application Publication
(10) Pub. No.: US 2026/0359947 A1
(43) Pub. Date: Nov. 12, 2026

(54) BLUE HALO REDUCTION UTILIZING LOCALLY VARYING BLUR

(71) Applicant: KARL STORZ IMAGING, INC., Goleta, CA (US)

(72) Inventor: Jonathan BORMET, Goleta, CA (US)

(21) Appl. No.: 19/202,104

(22) Filed: May 8, 2025

(51) Int. Cl. G06T 5/70; G06T 5/73

(52) U.S. Cl. CPC G06T 5/70; G06T 5/73; G06T 2207/10024; G06T 2207/10068; G06T 2207/20192

(57) ABSTRACT

The proposed exemplary technique is able to mitigate blue halos with locally varying spatial extent by performing a search over varying amounts of blur to apply to the green channel when sharpening the blue channel. Because the input blue channel is unmodified when forming Cb, the potential for gross artifacts is limited.

### FIELD

An exemplary aspect relates to reducing halos in images. More specifically, an exemplary aspect relates to reducing blue halos in images. Even more specifically, an exemplary aspect relates generally to reducing blue halos in images using locally varying blur.

### SUMMARY

According to one aspect, one goal aims to reduce chromatic aberration (CA) by first digitally blurring one color channel (which is at a better focus) until that first color channel approximately matches the optical blur of another color channel (which is significantly out of focus). Once aligned, the blurred channel is sharpened using content from the color channel having the better focus. This can be especially useful with medical imaging but is not limited thereto.

Rigid endoscopes for use with proximal camera heads tend to focus different wavelengths (or colors) of light at different focal planes. Oftentimes, when an object in the scene is at best focus for the green channel, the red channel is approximately focused but the blue channel is significantly out of focus (i.e., chromatic aberration). This results in the presence of a “blue halo” around edges in the scene in particular because the blue channel transitions over a larger portion of the image than the other color channels. Subsequent image processing of this image may amplify the problem and make the CA more visible.

As discussed herein, this problem is addressed by preprocessing an image by digitally blurring the green channel until the green channel approximately matches the optical blur of the blue channel. Once aligned, the blue channel is sharpened (i.e., unsharp masking) using content from the green channel. This can be done prior to other downstream image processing operations which may amplify the problem of haloing such as sharpening or tone mapping.

It is common in video processing pipelines for endoscopes/exoscopes to convert the red, green, and blue color channels obtained by a sensor, such as one in a camera/sensor, or the like, into a luminance-chrominance space such as YCbCr. Here, Y contains the grayscale component of the image and Cb and Cr encode the blue and red color differences, respectively.

Because the visual system is less sensitive to loss of detail in Cb and Cr, and because Cb and Cr often contain noise, it is common to low pass filter (digitally blur) the Cb and Cr channels.

However, if the green information in an image is sharp and blue information in the image is blurry, the Cb signal can include unwanted ringing artifacts and therefore any subsequent blurring of Cb will not necessarily resolve the ringing.

Assume that everything is linear, and thus Cb=B-G, and blur (Cb)=blur (B)-blur (G). One aspect works with this concept and assumes that the blue channel is optically blurred and the system digitally creates blur (G) such that the blur (Cb) term can be obtained directly.

Therefore, the blurriest channel can be used as a reference where, for example, the blurry blue channel is the reference since the technique uses a blurry Cb signal, since this signal is commonly required in video pipelines to use bandwidth efficiently and to reduce noise. But, the technique doesn't use just any blur (Cb), the technique uses the one formed where B and G have matching amounts of blur.

In accordance with one aspect, the amount of blur applied varies pixel to pixel within an acquired image. This is because the aberration in the scene can vary significantly based on the depth of the object I the scene or the type of image capture apparatus, i.e., endoscope, attached to the camera head. Thus, the technique can optionally adaptively blur G to match B locally, within a scene region. The technique doesn't necessarily have to directly align MTF, but the technique attempts to ensure that the Cb color difference signal is inherently low frequency in nature, and free of high frequency information which may include chromatic aberration ringing artifacts.

If the MTF (Modulation Transfer Function) of blue and green are aligned, the MTF would also have this property that the difference between the two color channels is small, and mostly low frequency.

One step in the disclosed technique is forming a stack of candidate images. The images in this stack are of the same scene but are of different blur amounts. One can think of it as similar to a scale space representation or a pyramid representation. However, the technique doesn't require downsampling as is typically done between layers of a pyramid. The technique can keep the blur images in the stack at the same size as the input image so the stack is not a pyramid.

FIGS. 2 and 3 described herein show the exemplary halo reduction benefits associated with the disclosed technology.

In accordance with another aspect, and in addition to blur, different types of chromatic aberration may manifest as image translation and magnification differences. Thus, in addition to searching in “scale space” over different blur amounts, a more general space incorporating translation and resizing could be used.

Furthermore, applying local, spatially varying amounts of image blur to mitigate blue halo/chromatic aberration problems would enable real-time, endoscopic application solutions to address haloing.

For example, and optionally in real or near-real-time, a camera controller could digitally blur the green channel using several Gaussian filters with varying spatial extent (varying a sigma parameter). A stack of candidate images is then formed (blue to green ratio images) using the input blue and digitally blurred green. Then the best halo-reduced pixel (or cluster of pixels) from each image in the stack is elected using, as one example, a scoring function such as a bandpass filter. With “best” ratio image pixels chosen at each spatial location as a subset of the pixels in the image stack, a corrected (sharpened) blue channel is produced.

Optional variants of the above scheme include interchanging the roles of which color channel is considered optically blurred and which color channel will be digitally blurred to match. And the system can make use of a Fourier frequency spectrum of the input channels to directly estimate the approximate amount of blur, while not explicitly forming a stack of images. This Fourier analysis could be done in a sliding window, i.e. locally for each pixel or for clusters of pixels.

Optionally still, aberration can also vary with the scene. One exemplary aspect continuously searches for a better or best correction to apply to every pixel or region on every frame.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The term “computer-readable medium” as used herein refers to any tangible storage and/or transmission medium that participate in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

The terms “determine”, “calculate” and “compute,” and variations thereof, as used herein, are used interchangeably and include any type of methodology, process, mathematical operation or technique.

It shall be understood that the term “means” as used herein shall be given its broadest possible interpretation in accordance with 35 U.S.C. § 112, Paragraph 6. Accordingly, a claim incorporating the term “means” shall cover all structures, materials, or acts set forth herein, and all of the equivalents thereof. Further, the structures, materials or acts and the equivalents thereof shall include all those described in any portion of this document. Any one or more of the embodiments herein can therefore be claimed using means-type language.

The preceding is a simplified summary of the disclosure to provide an understanding of some aspects of the disclosure. This summary is neither an extensive nor exhaustive overview of the disclosure and its various aspects, embodiments, and/or configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and/or configurations of the disclosure are possible utilizing, alone or in combination, one or more of the features set forth above or described in detail below.

These and other features and advantages of this technology are described in, or are apparent from, the following detailed description of the exemplary embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The exemplary embodiments will be described in detail, with reference to the following figures, wherein:

FIG. 1 illustrates an exemplary halo reduction system according to one aspect.

FIG. 2 illustrates an example rendered input image.

FIG. 2A illustrates an color example rendered input image.

FIG. 3 illustrates an exemplary blue halo reduced image from FIG. 2 that was processed according to the techniques disclosed here.

FIG. 3A illustrates an exemplary blue halo reduced image from FIG. 2A that was processed according to the techniques disclosed here.

FIG. 4 illustrates an exemplary method of improving image quality according to one aspect.

FIG. 5 illustrates an exemplary method of improving image quality according to one aspect.

FIG. 6A illustrates a raw image with optical CA.

FIG. 6B illustrates the RGB camera output per pixel of the raw image in FIG. 6A.

FIG. 6C illustrates the same image as in FIG. 6A post image processing with a visible blue halo.

FIG. 6D illustrates the same image as in FIG. 6C post image processing with a more visible blue halo.

FIG. 7A shows the effects of focal shift over wavelength.

FIG. 7B shows the effect of filtering at different wavelengths.

FIGS. 8-11 show graphs of edge profiles, the edge profile intensity range, a FFT of the edge profile and the resulting image in the region of interest as sigma varies.

FIGS. 12-15 show exemplary input images and details of those images before and after processing by the disclosed techniques.

FIG. 16 shows the sigma value of the gaussian blur chosen to apply locally within an exemplary input image.

FIGS. 17-18 show artificial biologic scenes where blue halos have been addressed in FIG. 18 using the techniques disclosed herein.

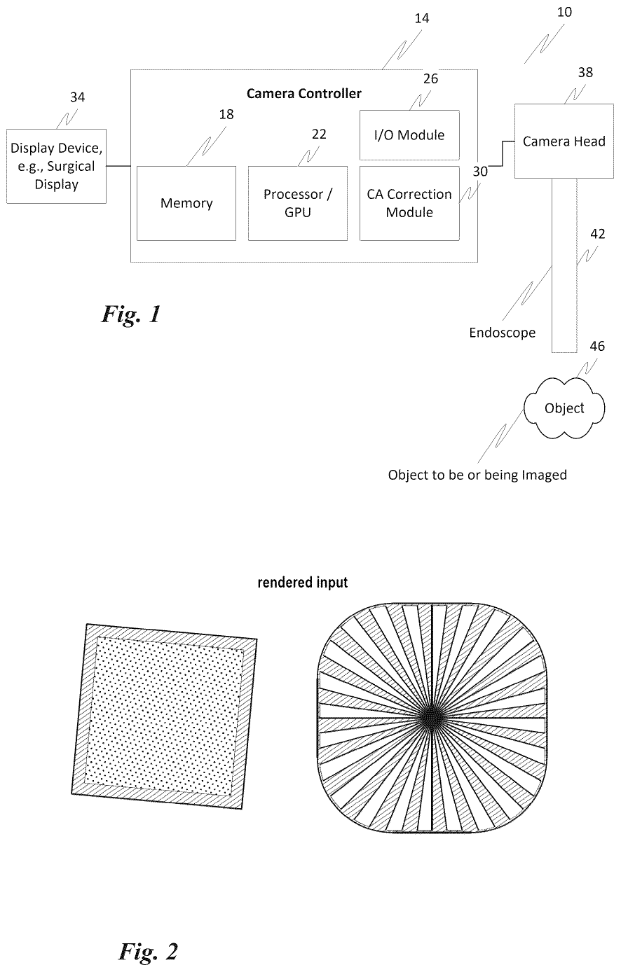

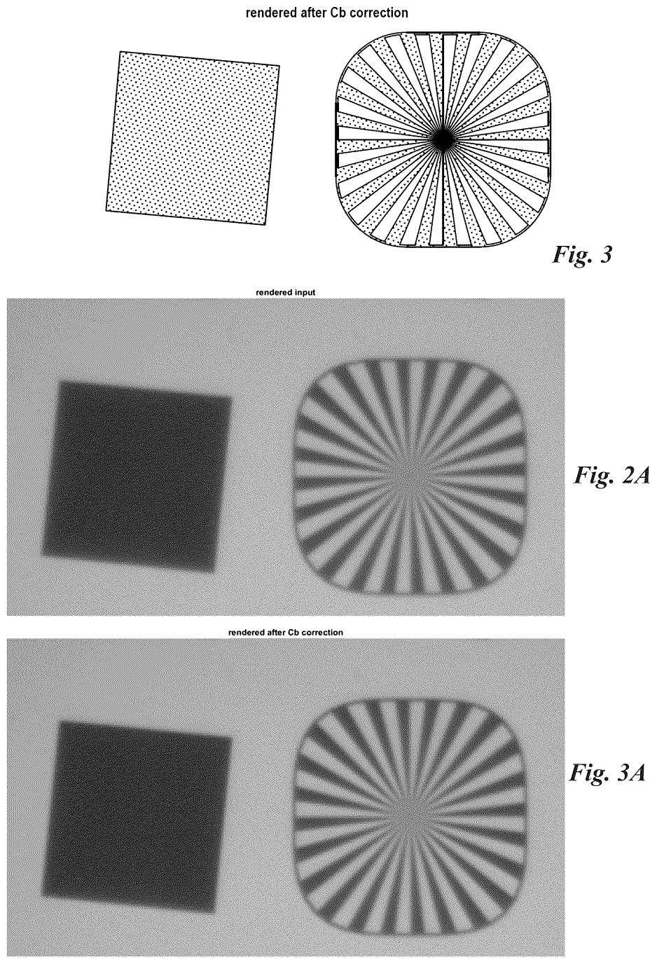

### DETAILED DESCRIPTION

The exemplary embodiments of this technology will be described in relation to digitally reducing chromatic aberration artifacts that can produce, for example, a blue halo around strong edges in an image.

For purposes of explanation, numerous details are set forth in order to provide a thorough understanding of the present technological solution. It should be appreciated however that the presently disclosed technology may be practiced in a variety of ways beyond the specific details set forth herein. Furthermore, while the exemplary embodiments illustrated herein show various components of this system collocated, it is to be appreciated that the various components of the system can be located at distant portions of a distributed or local network

Thus, it should be appreciated that the components of the system can be combined into one or more devices, or split between devices. As will be appreciated from the following description, and for reasons of computational efficiency, the components of the system can be arranged at any location within a distributed network or system without affecting the operation thereof. Similarly, one or more of the components could be included in a camera head or endoscope/exoscope.

As discussed, the disclosed technique suppresses the chromatic aberration artifact that produces a blue halo around strong edges. As this artifact is often amplified or exaggerated by tone mapping and sharpening operators (or other image processing techniques), one exemplary goal of the technique is to address the problem early in the pipeline.

Testing showed that for some endoscopes, the best focus for blue typically occurred at a significantly different focal plane than best focus for green. Red and green also diverge somewhat, but approximately agree. So in many portions of an endoscopic image, the red and green channels of the image will have similar sharpness, while the blue channel will be significantly blurred.

To correct this blurring, one aspect takes the optically sharp green channel and digitally blurs it using a Gaussian kernel of varying spatial extent (a sigma parameter) to form a stack of images. The optically blurred blue channel is used to form B:G ratios for each pixel (or cluster of pixels) in each blurred image in the stack. This linear ratio information is equivalent to a difference of logarithmic signals, which is similar in form to the chroma signal Cb commonly used in formats such as YCbCr. However, and throughout this disclosure “Cb” will refer to this B:G ratio, instead of the more typical definitions found in standards such as BT.709. Essentially the technique can be thought of using linear ratios rather than gamma corrected difference signals. Additionally, the technique approximates the luma channel Y as equal to G rather than the usual weighted average of R, G, and B. Typically and since G is the largest weighted component of Y, this approximation is reasonable for this approach. Thus, the technique assumes that green channel is used as if it is luminance.

In operation, some assumptions are made with one being that the approach presented herein should correct the channels in isolation before mixing to provide the greatest benefit. This can present a better output image with reduced haloing. Also, and as discussed, the technique will work for any color halo, but since blue is the dominant problem encountered in endoscopic imagery, the disclosure will focus, without limitation, on this color channel. Additionally, green will be used as the reference for this disclosure since it is closest to luminance and what a user will select for best focus.

With these assumptions in place, each pixel in each candidate Cb image formed from pairing a particular green blur amount with blue is scored for presence of ringing artifacts associated with a blue halo. This score may be determined by applying a bandpass filter to extract the power in lower frequencies (exclusive of the DC term that encodes average color).

With the Cb quality score for each pixel in a stack of Cb images, the best Cb value is chosen at each pixel by selecting the pixel from the Cb image with the best score (least power in the blue halo bandpass filter). This collapses the stack of Cb images into a single plane “best Cb” image.

This corrected Cb image is then used to form a corrected blue channel. Since the input Cb was the B:G ratio, the corrected B channel is formed by multiplying the input G by the corrected Cb in accordance with:

B_corrected=(Cb_best)*G_input

B_corrected=(B_input/G_best_blur)*G_input

This equation could optionally instead be implemented in logarithmic space, if desired. Note that by grouping the G blur and input terms, the operation can be understood as an unsharp mask applied to the blue channel, however the sharpening signal is extracted from the green channel rather than blue. An unsharp mask can increase the image contrast along the edges of objects. The mask image contains a higher intensity response near strong edges in the input image, but it does not explicitly detect and label edges. (Additional details regarding unsharp masking can be found here: https://en.wikipedia.org/wiki/Unsharp_masking, which is incorporated herein by reference.)

As another alternative, instead of searching over all candidate blur amounts and forming a stack of Cb images, a Fourier spectrum analysis of the input blue and green channels can be used to directly determine the “best” blur. Each frequency bin contains a different B:G power ratio which in turn corresponds to a specific Gaussian blur amount needed to align one channel to the other (at that frequency bin). However, estimates for the blur amount will vary bin to bin and could be combined into a weighted average blur amount. This is similar to the bandpass filter described herein.

As an alternative to directly implementing a Gaussian blur kernel at each sigma value, a scale space implementation (i.e., pyramid processing) can be used to coarsely sample the number of discrete blur images formed. Additional precision in blur amounts can be obtained by interpolating between these discrete blur images. Also, and instead of downsampling image content at each layer of the pyramid, the blur kernels may be upsampled to cover a larger region in the input image, but remain at the input resolution.

While, as discussed, blue halos are the most common and most objectionable form of chromatic aberration, these same techniques could be used to correct the Cr (red chroma) channel. Similarly, the assumed relationship of optically sharp green and optically blurry blue may be reversed in some portions of some input scenes. In that case, complimentary errors (i.e., yellow halos) may form. By considering potential digital blur amounts for all three color channels and considering all optically sharp/blurry channel pairings, a more general process could be used to locally correct the “worst” channel using the “best” channel in a region-by-region approach.

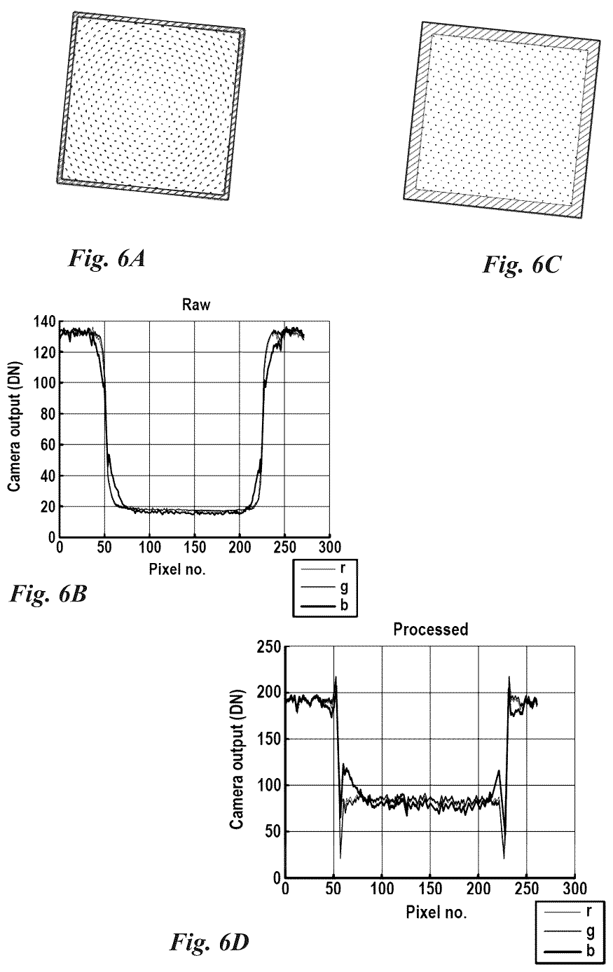

To put these techniques in more of a practical light, FIGS. 6A-B illustrate that CA issues originate as optical artifacts, but are amplified by image processing as shown in the processed images shown in FIGS. 6C-D. More specifically, FIG. 6A illustrates a raw image with optical CA. Here, haloing can be seen at least around the edges of the square in the RAW image. FIG. 6B illustrates the RGB camera output per pixel of the raw image in FIG. 6A. FIG. 6C illustrates the same image as in FIG. 6A post image processing with a more visible and amplified blue halo. FIG. 6D illustrates the RGB camera output per pixel of the same image as in FIG. 6A post image processing with the amplified more visible blue halo.

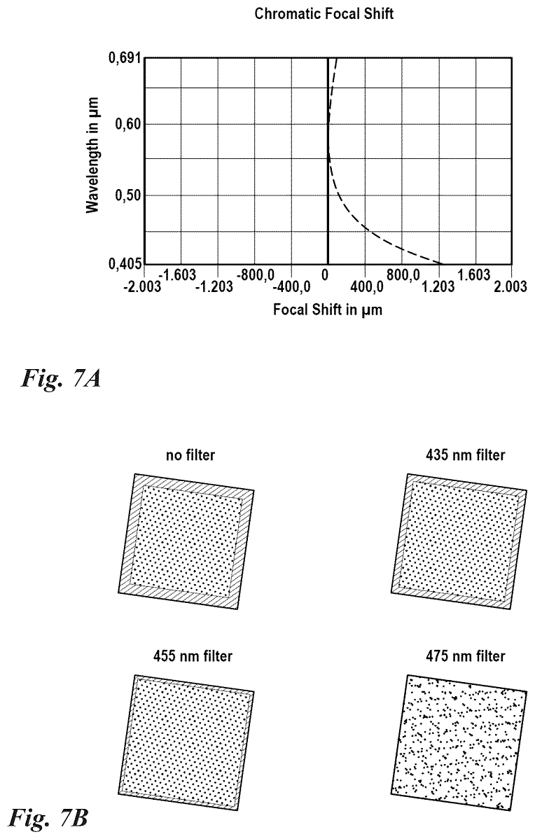

FIG. 7A-B show that if green wavelengths are at best focus, red is slightly defocused and blue is very defocused. Optically filtering out blue wavelengths mitigates the visibility of the focus error, but of course affects the color performance as well as seen in FIG. 7B with no filter in the upper left, a 435 nm filter upper right, a 455 nm filter lower left and a 475 nm filter lower right.

As introduced above, in image processing it is common to represent video signals in a luma/chroma space, where the Cb chroma signal is formed as a difference between gamma corrected B and Y signals, which is more or less equivalent to linear B to Y ratios. Since the human visual system is less sensitive to degradation of chroma signals, the chroma can be blurred and downsampled. However, if the blue channel is inherently much blurrier than the green channel, this Cb signal will resemble an unsharp mask sharpening operator near edges in the image. This observation forms the core of the correction technique disclosed herein. Specifically, and rather than forming Cb from blurred B and sharp G, the technique can form a corrected Cb by digitally blurring G to approximately match the optically blurred B channel.

The resulting signal comprised of two blurred inputs is of course still blurred, but this usually is intended for chroma signals. The luma signal Y is normally defined as an appropriately weighted average of the color components, but the technique disclosed herein simply treats G as Y. This is approximately correct, and avoids mixing color channels until the blur difference has been addressed.

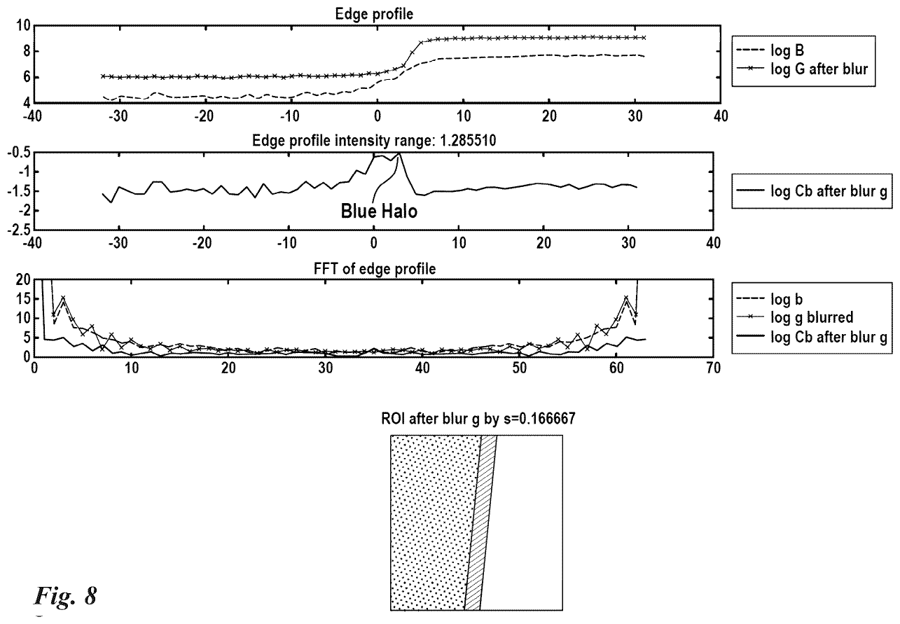

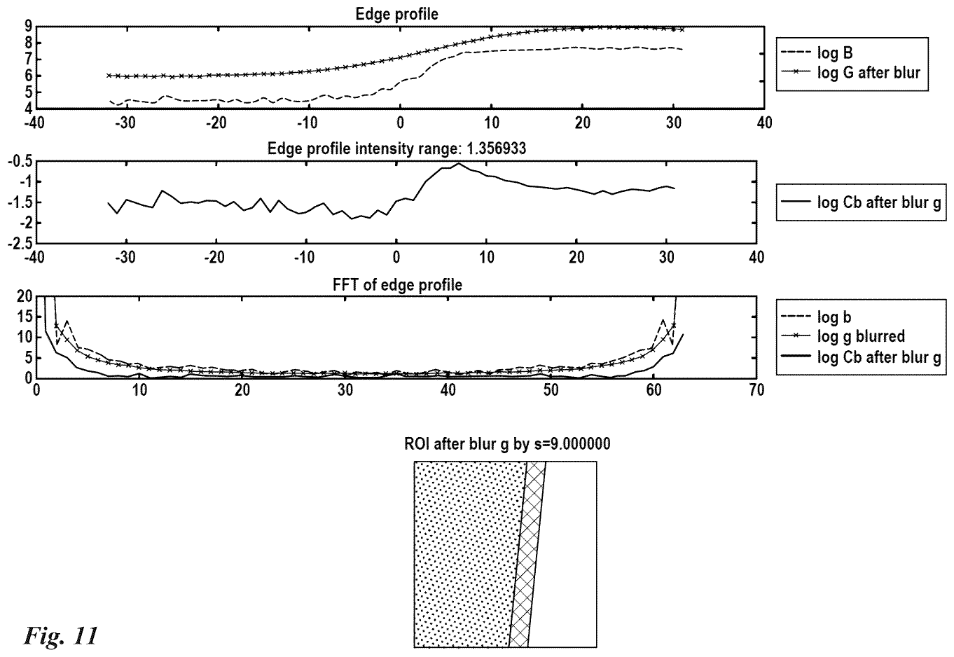

FIGS. 8-11 were produced by processing a raw image with the technique disclosed herein and show an example of an edge profile obtained from a slanted edge target. In each figure, the green, and only the green channel, has been blurred prior to forming Cb. Initially (no blur), Cb is roughly constant on either side of the edge but there is a significant peak in the vicinity of the edge, which is what produces the blue ringing artifact. As green is further blurred, the Cb peak comes down, and eventually reaches a local minimum around Gaussian blur sigma=3. Blurring green too much causes the Cb peak to reappear, this time introducing a yellowish fringe in the edge.

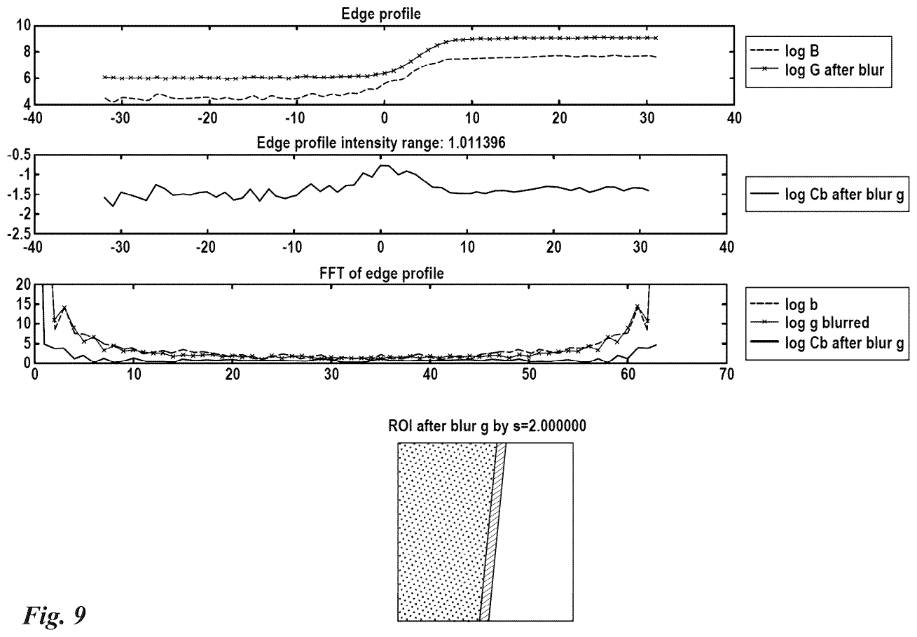

In FIG. 8, the spike in the center image corresponds to the blue halo. Then, in FIG. 9, where the Gaussian blue has been increased to 2, the green channel which was sharp was blurred to match the green. Here, using the FFT of the edge profile can provide results and the Gaussian blur is used with varying sigma such that there is a “sweet spot” where the blurred green approximately matches the naturally blurry blue channel.

FIGS. 9-11 continue to increase the sigma (from 0.166667 to 9.0, respectively) and illustrate the corresponding outputs. Specifically, example edge profiles were produced at varying levels of green blur (Note the varying sigma “s=####” in each figure). There is a “sweet spot” where the blurred green approximately matches the naturally blurry blue channel. The first subplot in each of FIGS. 8-11 shows the input blue channel and digitally blurred green channel. The second subplot shows the resultant Cb channel (where the spike in the middle is the halo). The third subplot shows the frequency spectrum of b, g, and Cb. And the fourth portion of the Figure shows a rendering of the region of interest (ROI) after blurring the green channel by the specified amount.

Examining the FFT of the edge profile, as shown in these figures, provides another view of the problem and solution. The original green channel is relatively sharp and shows the signatures of a square wave in the low frequency components. However, in the blue channel only the very lowest frequency bins contain this detail, and the spectrum quickly degrades. When subtracting the two channels, the residual Cb channel winds up having elevated low frequency content in this band where green and blue differ in sharpness. As green is incrementally blurred, the frequency content of the green channel begins to approximately align with the blue channel. When the two channels are in good agreement, the low frequency portion of Cb is suppressed. As green becomes over blurred, the low frequency content of Cb elevates again. Note that in this discussion “low frequencies” refers to the somewhat low frequencies where the blue channel provides spectral details but does not include the DC term itself. The DC term corresponds to the average color of the region and is not affected by this process.

In more detail, the exemplary process can be described in relation to the following. Specifically, the Cb correction technique will chose the “best” amount of blur to apply to the G signal from a range of candidate blur amounts. These candidate blur amounts can be predefined based on known results that provided an acceptable result, and/or can be determined on the fly, e.g., in real-time or near real-time. The amount of blur needed can vary locally throughout the image because scene objects will likely be at different depths and the spatial extent of the halo artifact will vary within an image because of this.

In order to select the “best” (from a human visual perception standpoint) amount of blur to apply to the green channel, the resulting Cb needs to be scored. The exemplary technique limits the low frequency content exclusive of DC, so a high pass filter formed from a very aggressive low pass filter can be used. (The signals will naturally have limited true high frequency content due to the bandlimited optics).

The sign of the signal components in this band is not important, only the magnitude or power. It is also undesirable to have wildly different amounts of blur applied pixel to pixel in the manner that might result if each pixel is independently optimized for its own blur score. This local smoothing, smoothness or normalization can be obtained by first extracting the high frequency power in a local neighborhood (that can be defined as any grouping of pixels), but then further smoothing this result with a low pass filter. This high pass-square-low pass filter structure is somewhat like a bandpass filter, but complicated by the nonlinearity in the middle. However, this flow approximately agrees with the visual inspection of the Fourier coefficients described in the edge profile figure shown previously.

The “best” sigma can be determined in accordance with the following:

$${σ}_{best} = {arg min}_{0 \leq σ \leq {σ}_{m ax}} LPF \{ {‖ HPF \{ log ( b ) - log ( {g}_{σ} ) \} ‖}^{2} \}$$

Where: Cb metric: the best Cb at each pixel is formed by blurring the green channel by the “best” sigma. This “best” sigma minimizes the Cb power in high frequencies, averaged over a local neighborhood. And a high pass filter of blue-green (sigma) where this is iterated from 0 to σₘₐₓ solves for the chroma with the least amount of high frequency energy.

Similar to the above, but expressed as logarithmic, green is blurred by varying amounts to form a stack of images somewhat like a scale-space representation without downsampling. A stack of Cb images is formed by differencing the logarithm of the input blue channel with each of the blurred green images. Then for each pixel the sigma that produces the lowest score for the power metric described above is chosen. Only the blurred green from the “best” sigma is used, effectively collapsing the stack down into a single “best” Cb image. This logspace Cb image is then converted back to linear B:G ratios, which are then applied to the blue channel similar to a gain map, essentially sharpening the blue channel using the green channel.

$${Cb}_{best} = log ( b ) - log ( {g}_{σ best} ) \\ log ( {b}_{corrected} ) = log ( g ) + {Cb}_{best} \\ log ( {b}_{corrected} ) = log ( g ) - log ( {g}_{σ best} ) + log ( b ) \\ {b}_{corrected} = g * exp ( {Cb}_{best} ) \\ {b}_{corrected} = \frac{g}{{g}_{σ best}} * b$$

As discussed, the correction can be thought of as a gain map applied to the blue channel (locally) to sharpen it using components of green. The “best” is determined as the stack of images with different amounts of blur appreciating that a better amount of blur can be determined and applied locally for a scene. The sigma that minimizes score is determined to be the best (and has the minimum residual energy).

Optional enhancements to this process include providing user selectable amounts of blur to select the best (human) visible output. This can be accomplished with, for example, a slider in a graphical user interface that shows before and after versions of the image (or local region).

Additionally, a slider (or comparable input mechanism) can be provided for the user to select/adjust the amount of blue to dial in and this could optionally be accomplished before image processing to assist with characterizing the system and honing the amount of blur.

Furthermore, while this technique has been discussed in relation to the blue channel, the technique could be applied as discussed to other channel(s) as well such as the red channel.

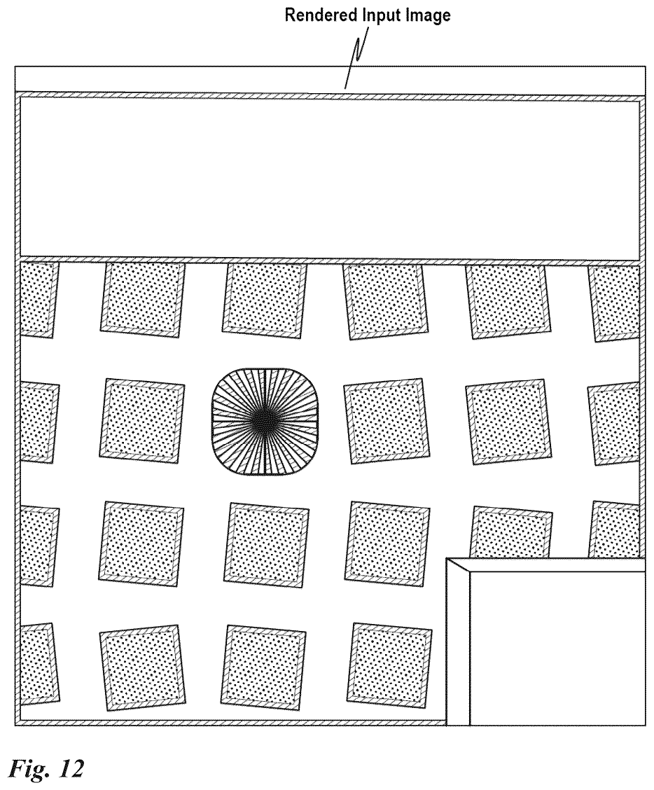

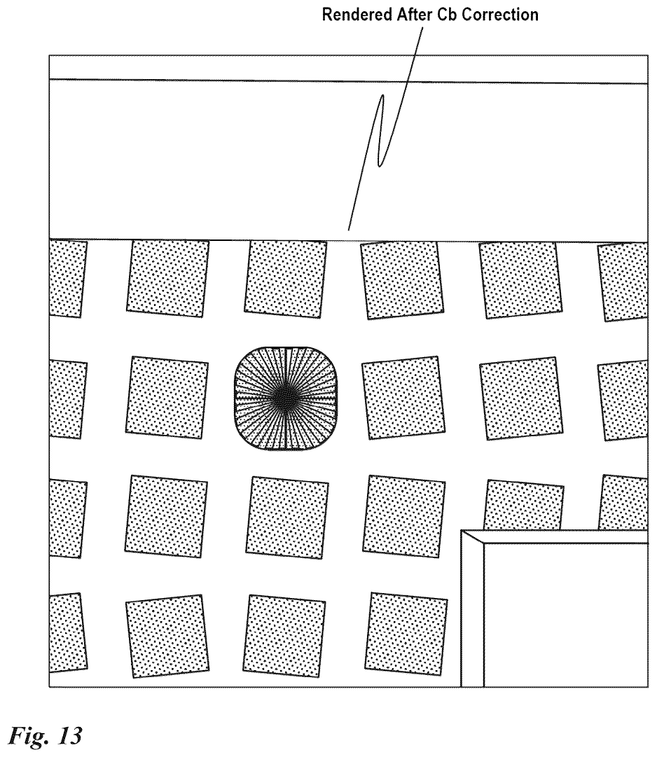

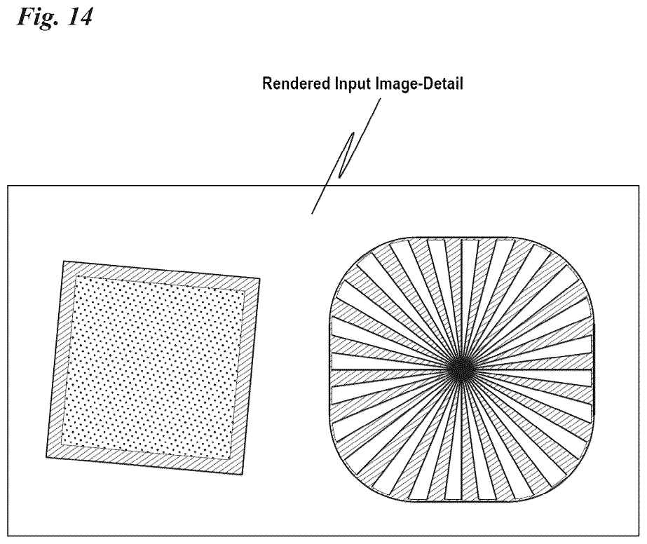

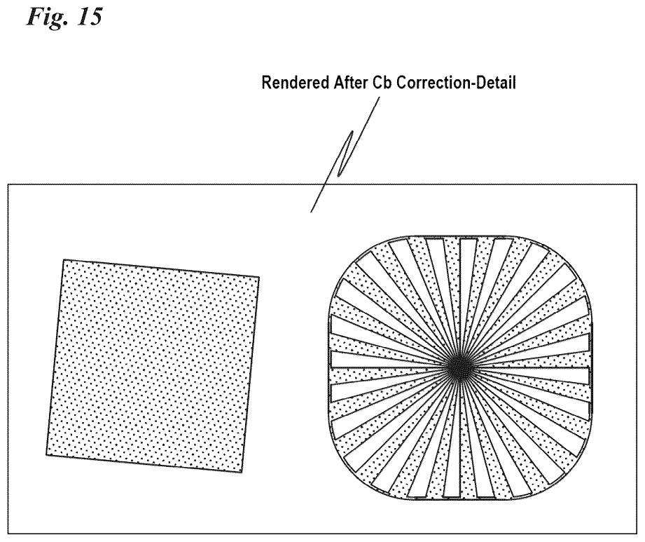

FIGS. 12-15 show a comparison of the input raw image (FIG. 12) and the rendered image after Cb correction (FIG. 13) with details of each shown in FIGS. 14 and 15, respectively.

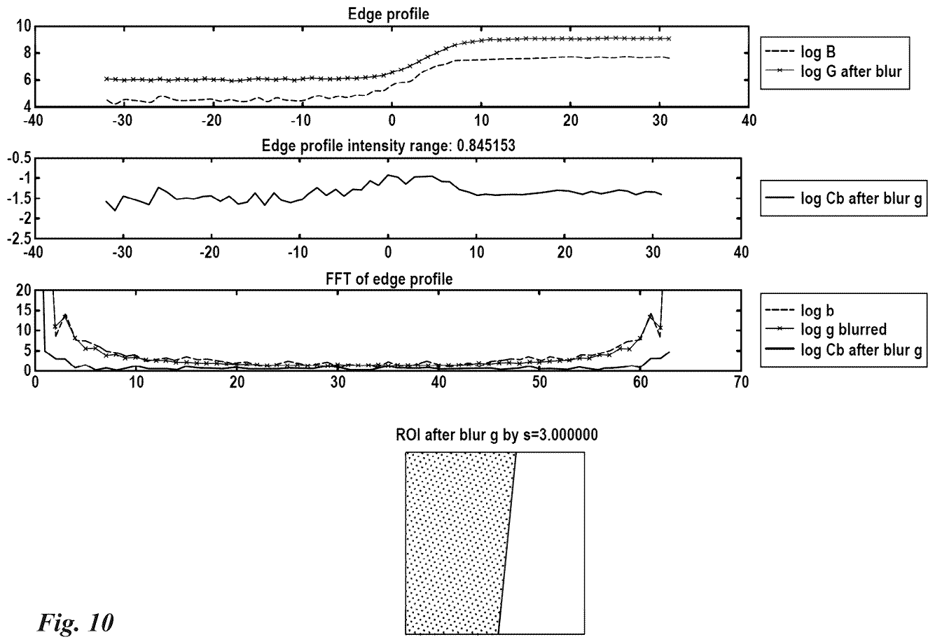

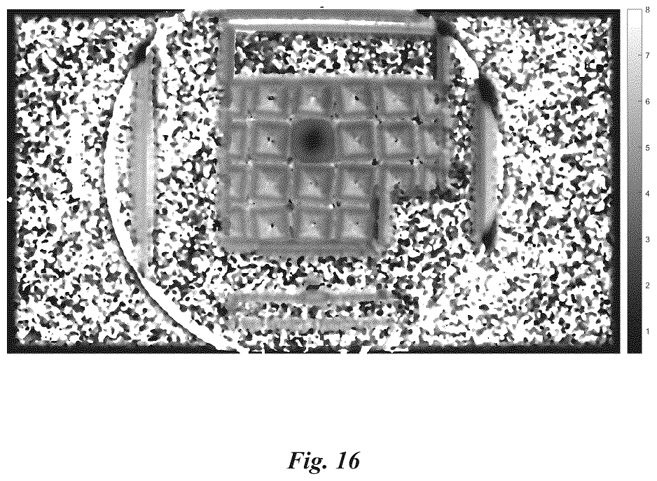

FIG. 16 shows the relationship of the sigma value used for each pixel. In areas with strong edges, the best sigma score tends to be a clear winner and the chosen value is stable in the vicinity of the edge feature. In areas with no texture, the metric and chosen sigma value can vary randomly. However, this does not introduce artifacts since these areas where both blue and green channels are flat do not change much visually no matter how much, or how little blur, is used to form Cb. In FIG. 16, 0 (black) is no blur, and 8 (white) is the most blurred. The amount of local blur can be seen based on the amount of white in local portions of the image. Of note, the strong edges produced by the slanted edge targets result in corresponding structures within the sigma value chosen to correct chromatic aberration. The gray level corresponding to a sigma of approximately 3 seen along the slant edge targets agrees with the flattest Cb edge profile shown in FIG. 10.

An optional feature that can be used with this technique is to perform edge detection first, as is known. (See for example: https://en.wikipedia.org/wiki/Edge_detection, which is incorporated herein by reference and also available in many commercial image processing applications.) Once the edges are detected, then the disclosed technique could process (locally) the information determined to be an edge. This could optionally improve performance.

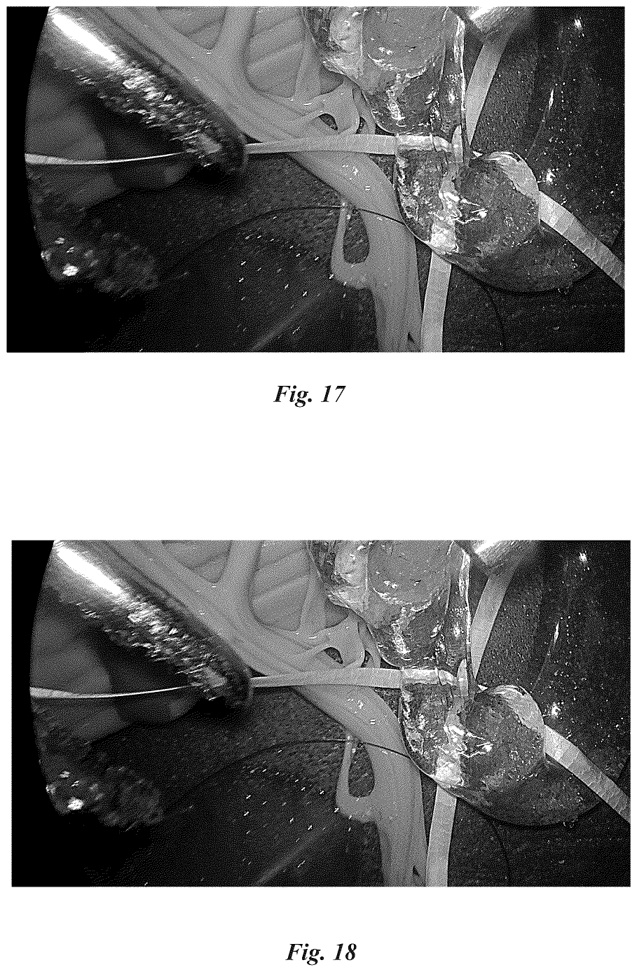

FIGS. 17-18 show artificial biological scenes where the techniques disclosed herein have been applied. FIG. 17 shows a representation of an original raw image, and FIG. 18 shows a representation of a rendered image where locally varying amounts of blue halo correction have been employed.

In this exemplary close-up of a scene, blue halos are present around specular highlights as well as the white ribbon material. The images are rendered with the techniques disclosed herein. The correction largely mitigates the most visually prominent halos. However, some blue remains around the large specular highlight in the top left. The yellow fringe at the edge of the scope outline has also not been corrected.

Natural extensions to this basic technique can include other blur relationships such as blurring G to form corrected Cr, or blurring B instead of G to correct yellow fringes, and the like.

Additional extensions include the avoidance searching over a range of candidate blur amounts and going directly to the sigma that optimizes the metric. This is possible since gaussian blurring in the spatial domain is equivalent to multiplying by a corresponding gaussian in the frequency domain. The ratio of the power at a given frequency bin in the input G and B channels “votes” for a particular attenuation factor (corresponding to a specific sigma value or blur amount) to align G to B at that bin. A weighted average of such blur votes can provide similar results to the searching scheme currently used.

In FIG. 1 an exemplary image processing system 10 is disclosed where well known components have been omitted for clarity. The system incudes a camera head 38 associated with endoscope/exoscope 42 that images an object 46. The camera head 38 outputs image/video information to the camera controller 14, and in particular the I/O module 26 and CA correction module 30.

The CA correction module 30 cooperates with the processor/GPU 22 and memory 18 to process images as discussed herein to generate and output a corrected image on display 34. As discussed, the display could also include a user interface that allows a user to select or vary an amount of blur to “dial in” to provide the best (to them) visual output of the system.

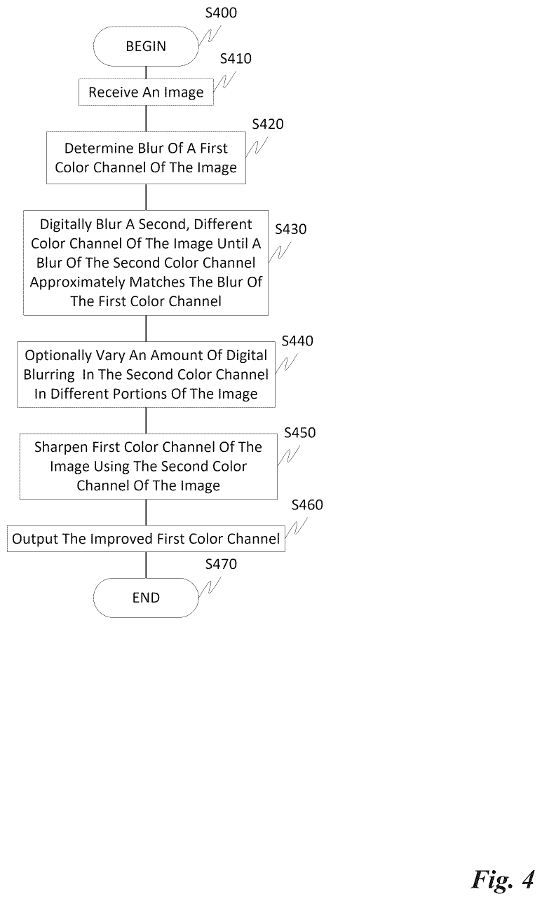

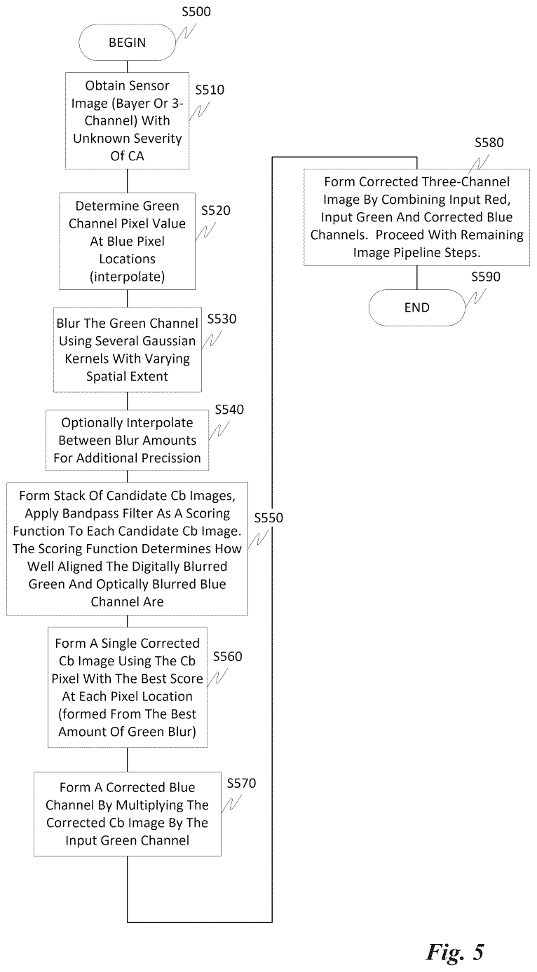

In operation, the image processing system 10 performs the general methods disclosed in FIGS. 4-5.

More specifically, and in a general operational mode, the image processing system 10 begins processing in step S400 and continues to step S410 where an image is received. Next, in step S420, a blur of a first color channel of the image is determined. Next, in step S430, the system digitally blurs a second, different color channel of the image until a blur of the second color channel approximately matches the blur of the first color channel. Then, and optionally in step S440, step S430 is iterated for different portions of the image such that a localized digital blurring is established and stored. As discussed, this can be done on a pixel-by-pixel basis, and/or on a cluster of pixels by cluster of pixels basis. Control then continues to step S450

In step S450, the first color channel of the image is sharpened based on the second color channel of the image. The resultant improved first color channel is then output optionally in combination with one or more other color channels. To assist the user, the display could also show the one or more sigmas that were used for the image. This could assist the user with selecting sigmas in the future for images with similar halo effects. Control then continues to step S470 where the control sequence ends.

In greater detail, FIG. 5 is a flowchart illustrating an exemplary method of operation of the image processing system 10. Specifically, control begins in step S500 and continues to step S510. In step S510, an image is obtained from a sensor, such as a Bayer or 3-channel sensor. This image will have an unknown amount of CA.

Next, in step S520, the green channel pixel value at blue pixel locations is determined through interpolation. Then, the green channel is blurred (digitally) using several Gaussian kernels having varying spacial extent. Control then continues to step S540.

In step S540, and optionally, interpolation between blur amounts can be performed to increase precision and accuracy. Next, in step S550, and after forming the stack of candidate Cb images, a bandpass filter is applied as a scoring function to each candidate Cb image. This scoring function determines or rates how well aligned the digitally blurred green and optically blurred channel are (in terms of blurring). Then, in step S560, a single corrected Cb image is generated using the Cb pixel with the best score at each pixel location (formed from the best amount of green blur). Control then continues to step S570.

In step S570 a corrected blue channel is formed by multiplying the corrected Cb image by the input green channel. Then, in step S580, a corrected three-channel image is output by combining the input red, the input green and the corrected blue channels. From here, the image can be further processed with the remaining image pipeline steps with control continuing to step S590 where the control sequence ends.

The systems, methods and protocols of this disclosure can be implemented on a special purpose computer, a programmed microprocessor or microcontroller and peripheral integrated circuit element(s), an ASIC or other integrated circuit, a digital signal processor, a flashable device, a hard-wired electronic or logic circuit such as discrete element circuit, a programmable logic device such as PLD, PLA, FPGA, PAL, an image processing device, an endoscope, a medical imaging device, or the like. In general, any device (or one or more equivalent means) capable of implementing a state machine that is in turn capable of implementing the methodology illustrated herein can be used to implement the various methods, protocols and techniques disclosed herein.

Furthermore, the disclosed methods may be readily implemented in software stored on a non-transitory computer-readable information storage media using, for example, object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosed technology is dependent on the speed and/or efficiency requirements of the system, the particular function, and the particular software or hardware systems or microprocessor or microcomputer systems being utilized. The systems, methods and protocols illustrated herein can be readily implemented in hardware and/or software using any known or later developed systems or structures, devices and/or software by those of ordinary skill in the applicable art from the functional description provided herein and with a general basic knowledge of the computer optical and endoscope arts.

Moreover, the disclosed methods may be readily implemented in software that can be stored on a computer-readable storage medium, executed on programmed general-purpose computer with the cooperation of a controller and memory, a special purpose computer, a microprocessor, or the like. The systems and methods of this technology can be implemented as a program embedded on personal computer such as an applet, JAVA® or CGI script, as a resource residing on a server or computer workstation, as a routine embedded in a dedicated system or system component, or the like. The system can also be implemented by physically incorporating the system and/or method into a software and/or hardware system, such as the hardware and software systems of an imaging device.

While this description is described in terms of exemplary embodiments, it should be appreciated that individual aspects of the technology could be separately claimed and one or more of the features of the various embodiments can be combined.

While the exemplary embodiments illustrated herein discuss the various components collocated, it is to be appreciated that the various components of the system can be located a distant portions of a distributed network, such as a telecommunications network and/or the Internet or within a dedicated communications network. Thus, it should be appreciated that the components of the system can be combined into one or more devices or collocated on a particular node of a distributed network, such as a communications network. As will be appreciated from the following description, and for reasons of computational efficiency, the components of the system can be arranged at any location within the distributed network without affecting the operation of the system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various aspects, embodiments, and/or configurations, includes components, methods, processes, systems and/or apparatus substantially as depicted and described herein, including various aspects, embodiments, configurations embodiments, subcombinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

It is therefore apparent that there has been provided, in accordance with the present disclosure, systems and methods for CA correction. While this disclosure has been described in conjunction with a number of embodiments, it is evident that many alternatives, modifications and variations would be or are apparent to those of ordinary skill in the applicable arts. Accordingly, this disclosure is intended to embrace all such alternatives, modifications, equivalents and variations that are within the spirit and scope of the disclosed technology.

1. A method to digitally reduce chromatic aberration in an image comprising:
receiving an image;
identifying a blur of a first color channel of the image;
digitally blurring a second, different color channel of the image until a blur of the second, different color channel approximately matches the blur of the first color channel of the image; and
outputting an improved first color channel.

2. The method of claim 1, wherein chromatic aberration is reduced in one or more of a red color channel, a green color channel and a blue color channel and an interpolation between amounts of blur increases accuracy.

3. The method of claim 1, further comprising sharpening the first color channel of the image using the second, different color channel of the image wherein a halo effect is reduced near object edges in the image.

4. The method of claim 1, wherein the image is an image stream from an endoscope.

5. The method of claim 1, wherein one or more of:
the method is performed in real-time on the received image, a final output comprises the improved first color channel, the second, different color channel and a third color channel, and
the first color channel is a blue color channel.

6. The method of claim 1, further comprising varying an amount of digital blurring of the second, different color channel of the image in different portions of the image.

7. The method of claim 1, wherein the blurring of the second, different color channel reduces power in high frequencies, in a ratio of the first and second color channels, that is averaged over a local neighborhood of pixels.

8. The method of claim 3, wherein the sharpening comprises multiplying a ratio of the input first color channel to the blurred second, different color channel by the second, different color channel.

9. The method of claim 1, wherein the approximate matching comprises using a Fourier frequency spectrum to estimate the amount of blur.

10. A system configured to digitally reduce chromatic aberration in an image comprising:
a camera control system, configured to be connected to a camera head, and configured to:
receive an image;
determine a blur of a first color channel of the image;
digitally blur a second, different color channel of the image until a blur of the second, different color channel approximately matches the blur of the first color channel of the image; and
output an improved first color channel.

11. The system of claim 10, wherein chromatic aberration is reduced in one or more of a red color channel, a green color channel and a blue color channel.

12. The system of claim 10, wherein the camera control system is further configured to sharpen the first color channel of the image using the second, different color channel of the image and a halo effect is reduced near object edges in the image.

13. The system of claim 10, wherein the image is an image stream from an endoscope.

14. The system of claim 10, wherein one or more of:
the outputting of the improved first color channel is performed in real-time on the received image, and a final output comprises the improved first color channel, the second, different color channel and a third color channel, and
the first color channel is a blue color channel.

15. The system of claim 10, wherein an amount of digital blurring of the second, different color channel of the image varies in different portions of the image.

16. The system of claim 10, wherein the blurring of the second, different color channel reduces power in high frequencies, in a ratio of the first and second color channels, that is averaged over a local neighborhood of pixels.

17. The system of claim 12, wherein the sharpening comprises multiplying a ratio of the input first color channel to the blurred second, different color channel by the second, different color channel.

18. The system of claim 10, wherein the approximate matching comprises using a Fourier frequency spectrum to estimate the amount of blur.

19. A non-transitory computer readable information storage medium having stored thereon instructions, that when executed by one or more processors, cause a method to digitally reduce chromatic aberration in an image to be performed comprising:
receiving an image;
determining a blur of a first color channel of the image;
digitally blurring a second, different color channel of the image until a blur of the second, different color channel approximately matches the blur of the first color channel of the image; and
outputting an improved first color channel.

20. The media of claim 19, further comprising sharpening the first color channel of the image using the second, different color channel of the image, wherein the blurring of the second, different color channel reduces power in high frequencies, in a ratio of the first and second color channels, that is averaged over a local neighborhood of pixels, and
wherein the sharpening comprises multiplying a ratio of the input first color channel to the blurred second, different color channel by the second, different color channel.
